(19) United States
(12) Patent Application Publication
(10) Pub. No.: US 2023/0114276 A1
(43) Pub. Date: Apr. 13, 2023

(54) MODULATING ANTI-TUMOR IMMUNITY

(71) Applicant: STICHTING HET NEDERLANDS KANKER INSTITUUT-ANTONI VAN LEEUWENHOEK ZIEKENHUIS, AMSTERDAM (NL)

(72) Inventors: Ruben Rowan LACROIX, AMSTERDAM (NL); Christian Ulrich BLANK, AMSTERDAM (NL)

(21) Appl. No.: 17/909,553

(22) PCT Filed: Mar. 3, 2021
(86) PCT No.: PCT/NL2021/050142

§ 371 Date: Sep. 6, 2022

(30) Foreign Application Priority Data
Mar. 6, 2020 (NL) 2025078

(51) Int. Cl. A61K 38/16; A61K 39/395; A61P 35/00

(52) U.S. Cl. CPC A61K 38/164; A61K 39/3955; A61P 35/00; A61K 2039/505

(57) ABSTRACT

The current invention relates to the treatment of cancer. In particular the invention relates to modulating the anti-tumor immunity in a cancer patient. The disclosed invention is in particular useful in the treatment of so-called cold tumors and/or in tumors that are resistant to or acquired resistance to treatment with immune checkpoint modulators. Compounds for use in the disclosed treatment, combinations and methods of treatment are provided.

### BACKGROUND OF THE INVENTION

The background description includes information that may be useful in understanding the present invention. It is not an admission that any of the information provided herein is prior art or relevant to the presently claimed invention, or that any publication specifically or implicitly referenced is prior art.

The promising results achieved in the past few years with the research on cancer immunotherapies and checkpoint inhibitors have revolutionized the field and have established the host immune response as a target for therapeutic anti-cancer treatments. Several immunotherapies aimed at enhancing the immune system of a cancer to target tumors in the patient have been developed and various others are under development. For example, the discovery of immune checkpoint receptors that repress the activity of anti-tumor T cells, such as PD-1, LAG-3, TIM-3, and CTLA4, led to the development of antibodies directed against these co-inhibitory receptors or their ligands (e.g. PD-L1 for PD-1). Clinical established examples include ipilimumab, pembrolizumab, nivolumab, spartalizumab, durvalumab, and atezolizumab. The value of cancer immunotherapy is evident from the fact that a part of the patients treated with checkpoint inhibitors experience durable tumor regression. This is in contrast to treatments based on small molecules where often tumor relapse is observed.

Despite these promising results unfortunately cancer immunotherapy is not successful in most cancer patients and response rates remain in most case at low percentages. It is believed that there are several distinct mechanisms underlying the observed limited response to cancer immunotherapy and the scientific community is just starting to unravel these mechanisms.

A major factor believed to be involved in initial resistance of a tumor to cancer immunotherapy, and in particular immune checkpoint inhibitors, is the lack of tumor T cell infiltration. Mechanisms involved in the absence of T cell infiltration, including lack of plethora tumor antigens, defects in antigen presentation, absence of T cell activation and deficit of homing into the tumor bed rates are being identified (Bonaventura et al. (2019) Front. Immunol., doi.org/10.3389/fimmu.2019.00168). Patient tumors that display such lack of or reduced T cell infiltration are referred to in the field as so-called “non-inflamed” or “cold tumors” (Galon et al. (2019) Nature Reviews Drug Discovery 18: 197-218, doi:10.1038/s41573-018-0007-y; Vareiki et al. (2018) J . Immunother Cancer 6(1):157, doi: 10.1186/s40425-018-0479-7). Specific treatments to overcome such absence of/reduced T cell infiltration have been suggested and include, amongst others, the use of PRR agonists, CD40 antibodies, TGF-beta blocking antibodies or receptor antagonists (Bonaventura et al. (2019) Front. Immunol., doi.org/10.3389/fimmu.2019.00168).

Furthermore, it is now accepted that, as in the case of targeted therapies, tumors can acquire resistance against immunotherapies (Restifo et al (2016) Nat Rev Cancer. 6(2): 121-126., doi: 10.1038/nrc.2016.2.). It is thought to be mediated by loss of MHC molecules or defects in the interferon responsiveness (Zaretzky et al N Engl J. Med. 375:819-829., doi: 10.1056/NEJMoa1604958), but are found in only minority of resistant tumors. Thus the vast majority of resistance to cancer immunotherapy is not understood yet.

In light of this, new products, compositions, methods and uses in the treatment of cancer are highly desirable. In particular, there is a clear need in the art for reliable, efficient and reproducible products, compositions, methods and uses that may broaden the type and number of tumors that respond to cancer immunotherapy and/or that further enhance the efficacy of cancer immunotherapy of existing approaches. Accordingly, the technical problem underlying the present invention can been seen in the provision of such products, compositions, methods and uses for complying with any of the aforementioned needs. The technical problem is solved by the embodiments characterized in the claims and herein below.

### Description

### DRAWINGS

Embodiments of the invention are further described hereinafter with reference to the accompanying figures:

FIG. 1: The effect of SEB on T cell infiltration in a “cold” melanoma. (a) Spontaneous tumors or tumors induced on the flank of Tyr::CreER^{T2}; Pten^{LoxP/LoxP}; Braf^{CA/+} mice with 4-OH tamoxifen were used in this experiment. Established tumors were injected with vehicle or 50 μg SEB. 48 hour after treatment tumors were formalin fixed and paraffin embedded and stained for CD3. (b) Infiltration was scored in three categories: “absent”, “low” and “high”. (c) Scoring of a minimum of 10 independent tumors.

FIG. 2: Effect of SEB plus immune checkpoint blockade in a “cold” melanoma.

(a) Tumors were induced on the flank of Tyr::CreER^{T2}; Pten^{LoxP/LoxP}; Braf^{CA/+} mice with 4-OH tamoxifen. Treatment was started when tumors reached 100-200 mm² (b) Pictures of all mice that were part of the experiment.

FIG. 3: Effect of SEB plus immune checkpoint blockade in a “hot” but immune refractory melanoma. (a) C57BL/6 mice were inoculated with 0.3×10⁶ cells. Tumor size was followed with a caliper twice weekly for the entire duration of the experiment. Mice were randomized into treatment groups when tumors reached ˜200 mm³.(b) Kaplan-Meier survival plot of the experiment.

FIG. 4: Effects if SEA on T cell infiltration. a) and b) tumor injected with SEA shows a remarkable increase in number of CD3+ T cells and CD3+CD8+ T cells. c) tumor injected with SEA showed a high CD3+ T cell and CD8+T cell infiltration throughout the tumor, compared with mice injected with vehicle control.

### Definitions

A portion of this disclosure contains material that is subject to copyright protection (such as, but not limited to, diagrams, device photographs, or any other aspects of this submission for which copyright protection is or may be available in any jurisdiction.). The copyright owner has no objection to the facsimile reproduction by anyone of the patent document or patent disclosure, as it appears in the Patent Office patent file or records, but otherwise reserves all copyright rights whatsoever.

Various terms relating to the methods, compositions, uses and other aspects of the present invention are used throughout the specification and claims. Such terms are to be given their ordinary meaning in the art to which the invention pertains, unless otherwise indicated. Other specifically defined terms are to be construed in a manner consistent with the definition provided herein. Although any methods and materials similar or equivalent to those described herein can be used in the practice for testing of the present invention, the preferred materials and methods are described herein.

For purposes of the present invention, the following terms are defined below.

As used herein, the singular form terms “a,” “an,” and “the” include plural referents unless the content clearly dictates otherwise. Thus, for example, reference to “a cell” includes a combination of two or more cells, and the like. For example, a method for administrating a drug includes the administrating of a plurality of molecules (e.g. 10's, 100's, 1000's, 10's of thousands, 100's of thousands, millions, or more molecules). As used herein, “and/or” refers to a situation wherein one or more of the stated cases may occur, alone or in combination with at least one of the stated cases, up to with all of the stated cases.

As used herein, “at least” a particular value means that particular value or more. For example, “at least 2” is understood to be the same as “2 or more” i.e., 2, 3, 4, 5, 6, 7, 8, 9, 10, 11, 12, 13, 14, 15, . . . , etc. As used herein, the term “at most ” a particular value means that particular value or less. For example, “at most 5” is understood to be the same as “5 or less” i.e., 5, 4, 3, . . . −10, −11, etc.

As used herein, “comprising” or “to comprise” is construed as being inclusive and open ended, and not exclusive. Specifically, the term and variations thereof mean the specified features, steps or components are included. These terms are not to be interpreted to exclude the presence of other features, steps or components. It also encompasses the more limiting “to consist of”.

As used herein, “conventional techniques” or “methods known to the skilled person” refer to a situation wherein the methods of carrying out the conventional techniques used in methods of the invention will be evident to the skilled worker. The practice of conventional techniques in molecular biology, biochemistry, cell culture, genomics, sequencing, medical treatment, pharmacology, immunology and related fields are well-known to those of skill in the art. and are discussed, in various handbooks and literature references. As used herein, “exemplary” means “serving as an example, instance, or illustration,” and should not be construed as excluding other configurations disclosed herein.

As used herein, “agonist”, as used herein refers to a compound or agent having the ability to initiate or enhance a biological function of a target protein or polypeptide, such as increasing the activity or expression of the target protein or polypeptide.

Accordingly, “agonist” is defined in the context of the biological role of the target protein or polypeptide. While some agonists herein specifically interact with (e.g., bind to) the target, compounds and/or agents that initiate or enhance a biological activity of the target protein or polypeptide by interacting with other members of the signal transduction pathway of which the target polypeptide is a member are also specifically included within this definition.

As used herein, “antagonist” and/or “inhibitor” are used interchangeably, and they refer to a compound or agent having the ability to reduce or inhibit a biological function of a target protein or polypeptide, such as by reducing or inhibiting the activity or expression of the target protein or polypeptide. Accordingly, the terms “antagonist” and “inhibitor” are defined in the context of the biological role of the target protein or polypeptide. While some antagonists herein specifically interact with (e.g., bind to) the target, compounds that inhibit a biological activity of the target protein or polypeptide by interacting with other members of the signal transduction pathway of which the target protein or polypeptide are also specifically included within this definition.

As used herein, “cancer” refers to the physiological condition in mammals that is typically characterized by unregulated cell growth. The terms “cancer,” “neoplasm,” and “tumor,” are often used interchangeably to describe cells that have undergone a malignant transformation that makes them pathological to the host organism. Primary cancer cells can be distinguished from non-cancerous cells by techniques known to the skilled person. A cancer cell, as used herein, includes not only primary cancer cells, but also cancer cells derived from such primary cancer cell, including metastasized (secondary) cancer cells, and cell lines derived from cancer cells. Examples include solid tumors and non-solid tumors or blood tumors. Examples of cancers include, without limitation, leukemia, lymphoma, sarcomas and carcinomas (e.g. colon cancer, pancreatic cancer, breast cancer, ovarian cancer, prostate cancer, lung cancer, melanoma, lymphoma, non-Hodgkin lymphoma, colon cancer, (malignant) melanoma, thyroid cancer, papillary thyroid carcinoma, lung cancer, non-small cell lung carcinoma, and adenocarcinoma of lung.). Treatment of a cancer in a subject includes the treatment of a tumor in the subject.

As used herein, an “effective amount” means the amount of a drug which is effective for at least a statistically significant fraction of subjects to treat any symptom or aspect of the cancer within the context of the treatment according to the invention. Effective amounts can be determined routinely. The term includes both pharmacological effectiveness and physiological safety. Pharmacological effectiveness refers to the ability of the treatment to result in a desired biological effect in the subject such as improvement of symptoms, a cure, a reduction in disease load, reduction in tumor mass or cell numbers, extension of life, improvement in quality of life, or other effect generally recognized as positive by medical doctors familiar with treating the particular type of disease or condition. Physiological safety refers to the level of toxicity, or other adverse physiological effects at the cellular, organ and/or organism level (often referred to as side-effects) resulting from administration of the treatment.

As used herein, a “modulator” refers to a compound that is either an agonist or an antagonist of the targeted protein.

As used herein, “resistant cancer” or “resistant tumor” refer to when a cancer or tumor that has a reduced responsiveness to a treatment, e.g., up to the point where the cancer does not respond to treatment. The cancer can be resistant at the beginning of treatment, or it may become resistant during treatment (acquired resistance). Acquired resistance indicates that a cancer or tumor has acquired reduced sensitivity or has become resistant to the effects of a drug or treatment after being exposed to it, or to a drug or treatment targeting the same mechanism or pathway, for a certain period of time. Acquired resistance to the therapy with a drug often manifests either a diminished amount of tumor regression for the same dose of a drug or the need for an increased dose for an equal amount of tumor regression.

As used herein, a “subject” is to indicate the organism to be treated e.g. to which administration is contemplated. The subject may be any subject in accordance with the present invention, including, but not limited to humans, males, females, infants, children, adolescents, adults, young adults, middle-aged adults or senior adults and/or other primates or mammals. Preferably the subject is a human patient. A subject may have been diagnosed with a cancer, or be suspected of having a cancer.

As used herein, “treatment”, “treating”, “palliating”, “alleviating” and “ameliorating” all refer to an approach for obtaining beneficial or desired results including, but not limited to, therapeutic benefit. By therapeutic benefit is meant eradication or amelioration of the underlying disorder being treated. Also, a therapeutic benefit is achieved with the eradication or amelioration of one or more of the physiological symptoms associated with the underlying disorder such that an improvement is observed in the patient, notwithstanding that the patient can still be afflicted with the underlying disorder.

As used herein, “simultaneous administration” refers to administration of more than one drug at the same time, but not necessarily via the same route of administration or in the form of one combined formulation. For example, one drug may be provided orally whereas the other drug may be provided intravenously during a patients visit to a hospital. “Separate administration” includes the administration of the drugs in separate form and/or at separate moments in time, but again, not necessarily via the same route of administration. “Sequentially” of “sequential administration” indicates that the administration of a first drug if followed, immediately or in time, by the administration of the second drug, but again, not necessarily via the same route of administration.

### DETAILED DESCRIPTION

It is contemplated that any method, use or composition described herein can be implemented with respect to any other method, use or composition described herein. Embodiments discussed in the context of methods, use and/or compositions of the invention may be employed with respect to any other method, use or composition described herein. Thus, an embodiment pertaining to one method, use or composition may be applied to other methods, uses and compositions of the invention as well.

As embodied and broadly described herein, the present invention is directed to the surprising finding that tumors that are unresponsive to, or only show limited response to, immunotherapy, in particular tumors that are unresponsive to, or only show limited response to, immune checkpoint modulators, become responsive to such treatment upon treatment with a Staphylococcal enterotoxin. In particular it was found that tumors that lack or have very low T cell infiltration (e.g. so-called “cold tumors) were highly infiltrated with T cells after exposure of the tumor to a Staphylococcal enterotoxin. Tumor growth and size of tumors initially unresponsive to immunotherapy, in particular to treatment with immune checkpoint modulators, was dramatically reduced after treatment with the combination of a Staphylococcal enterotoxin and an immune checkpoint inhibitor.

Therefore, according to an aspect of the current invention, there is provided for Staphylococcal enterotoxin, or biological active fragments thereof, for use in the treatment of cancer in a subject having a tumor, wherein the treatment further comprises administration of an immune checkpoint modulator to the subject.

Staphylococcal enterotoxins (SEs) relate to members of a family of more than 20 different staphylococcal and streptococcal toxins that are functionally related and share sequence homology (see for an extensive review Pinchuk et al (2010) Toxins (Basel). 2(8): 2177-2197, doi: 10.3390/toxins2082177 and Krakauer et al (2013) Virulence 15; 4(8): 75-773, doi: 10.4161/viru.23905). These bacterial proteins are known to be pyrogenic and are connected to significant human diseases that include food poisoning and toxic shock syndrome. These toxins are for the most part produced by Staphylococcus aureus (S. aureus) although other species have also been shown to be enterotoxigenic.

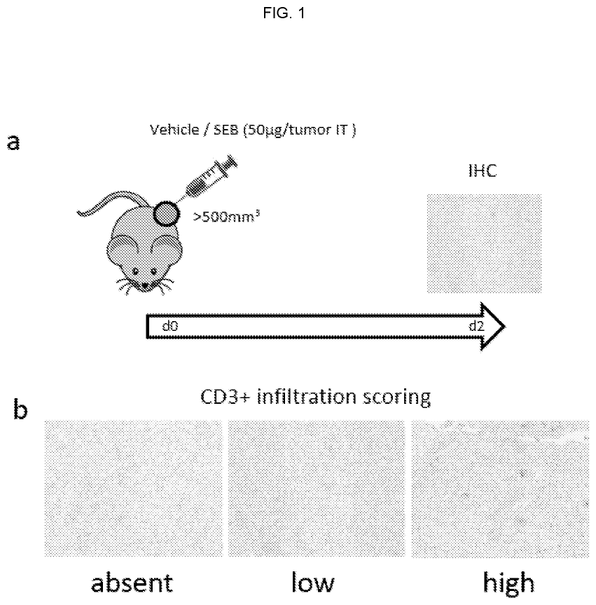

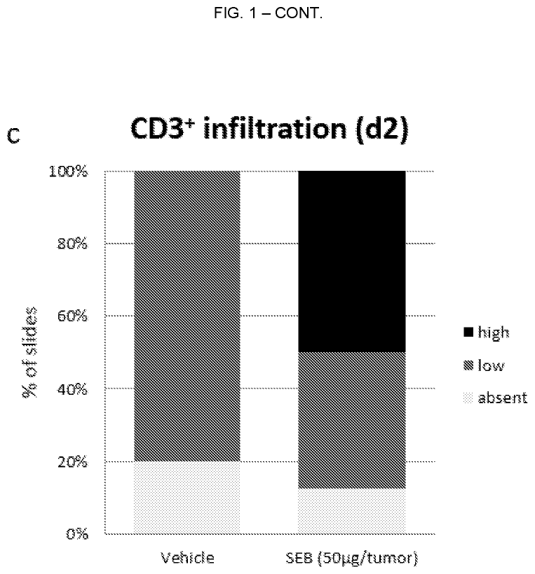

At least 20 serologically distinct natural staphylococcal enterotoxins have been described, that include SEs A through V, e.g. SEA, SEB, SEC (including C1, C2, C3), SED, SEE, and SEF, SEG, SEI, SEMM, SEN, SEO, SEQ and SEU. For example Staphylococcal enterotoxin A (SEA), SED, and SEE share 70-90% sequence homology, and 40-60% with SEB, and SEC. The different natural staphylococcal enterotoxins are conserved in a so-called b-strand(8)/hinge/a-helix(4) toxin domain (see, for example Arad et al. (2000) Nat Med; 6(4):414-21. doi: 10.1038/74672 or (Popugailo A., et al., (2019) Front. in Imm. vol. 10(942): p. 2, FIG. 1)), expected to be relevant for biological activity of the various staphylococcal enterotoxins (including SE A-V, for example SEA, SEB, SEC, SEA, SED, SEE, SEG, SEH, SEI, SEJ). The mature length of the SEs is approximately 220-240 amino acids, depending on the toxin, and their molecular size is on average ˜25 kD. The complete amino acid composition of various of staphylococcal enterotoxins has been reported (see e.g., PCT Patent Appl. No. WO 93/24136 and/or e.g. www.uniprot.org/uniprot/A0A0H2WZB2

In addition to the above-described natural occurring SEs, various biological active fragments, chemical derivatized forms, and genetically modified forms of these SEs are known. Such biological fragments, chemical derivatized forms, and/or genetically modified forms are for example described for SEB (Gu, L., et al., (2013) PLoS One, 8(): p. e55892.; Yousefi, F., et al., (2016) Tumour Biol, 37(4): p. 5305-16; Lansley, S. M., et al., (2014) Respirology, 19(7): p. 1025-33.), SEA (Jeudy, G., et al., (2008) Cancer Gene Ther, 15(11): p. 742-9.; Giantonio, B. J., et al. (1997) J Clin Oncol, 15(5): p. 1994-2007; Alpaugh, R. K., et al. (1998), Clin Cancer Res, 4(8): p. 1903-14.; Takemura, S., et al. (2002), 51(1): p. 33-44.; Golob-Urbanc, A., et al. (2019) J Biol Chem, 294(16): p. 6294-6305) and other SEs (Zhao, W., et al. (2016) Toxins (Basel), 8(6).; Wang, X., et al. (2009) Cancer Immunol Immunother, 58(5): p. 677-86.; He, Y., et al. (2019) Med Microbiol Immunol, 208(6): p. 781-792).

With respect to the current invention, such genetically modified forms, chemical derivatized forms, and/or biological active fragments from such natural or genetically modified SEs are to be understood to be included under the term Staphylococcal enterotoxin.

According to the current disclosure, the Staphylococcal enterotoxin are useful in the treatment of cancer in a subject having a tumor, wherein the treatment further comprises administration of an immune checkpoint modulator to the subject. As can be witnessed from the Examples, the Staphylococcal enterotoxin improves, enhances, strengthens, and/or increases the ability of the immune system (T cells) to infiltrate a tumor and impose an anti-cancer response in combination with immune checkpoint modulators.

It will be appreciated by the skilled person that treatment with an Staphylococcal enterotoxin and an immune checkpoint modulator now allows for the treatment of tumors that are unresponsive to treatment with immune checkpoint inhibitors alone. It will also be appreciated by the skilled person that the treatment with an Staphylococcal enterotoxin and an immune checkpoint modulator as disclosed herein allows for the treatment of tumors that are resistant to, or that have acquired resistance to the treatment with immune checkpoint modulators (so-called immune checkpoint modulator (acquired) resistant tumors or cancers). In addition, it will be appreciated that treatment of an tumor with a Staphylococcal enterotoxin and an immune checkpoint inhibitor allows to enhance the response to immune checkpoint modulators also in tumors that are—to a certain degree—already responsive to an immune checkpoint modulator alone. It will thus be appreciated by the skilled person that the disclosed treatment with an Staphylococcal enterotoxin and an immune checkpoint modulator is useful in the treatment of in principal any kind of tumor/cancer.

The treatment according to the invention further comprises administration of an immune checkpoint modulator to the subject, i.e. as part of an immune checkpoint therapy.

An “immune checkpoint therapy” refers to the use of agents that modulate immune checkpoint nucleic acids and/or proteins. Modulation of one or more immune checkpoints can block or otherwise neutralize inhibitory signaling or enhance or otherwise increase stimulatory signaling to thereby upregulate an immune response in order to more efficaciously treat cancer.

Exemplary agents useful for modulating (inhibiting or stimulating; immune checkpoint modulators) immune checkpoints include antibodies, small molecules, peptides, peptidomimetics, natural ligands, and derivatives of natural ligands, that can either bind, inactivate or inhibit, or active or stimulate, immune checkpoint proteins, or fragments thereof; as well as RNA interference, antisense, nucleic acid aptamers, etc. that can downregulate or upregulate the expression and/or activity of immune checkpoint nucleic acids, or fragments thereof. Exemplary agents include agents that block the interaction between inhibitory immune checkpoint proteins and its natural receptor(s) and/or agent that stimulate interaction between stimulatory immune checkpoint proteins and its natural receptor(s).

As used herein “immune checkpoint” refers to a group of molecules expressed on the cell surface of (CD4+ and/or CD8+) T cells, other immune cells, or on target cells (including tumor cells) that fine-tune immune responses by either turning up a signal (also known as “stimulatory immune checkpoints”) or turn down a signal (also known as “inhibitory immune checkpoints”), thereby modulating an anti-tumor immune response.

Immune checkpoint proteins and pathways are well-known in the art and include, without limitation PD-1, PD-L1, PD-L2, CTLA-4, LAG3, B7-H3, B7-H4, B7-H5 (VISTA), KIR, TIGIT, CD47, TIM3, CD70, CD27, CD160, CD112, CD134 (OX40), CD226, CD155, A2aR, CD40, CD40L, CD137, CD270, CD272, CD275, CD278, CD28, GITR (TNFS18), B7-H2, B7-H6, HVEM, B7.1, TIM-1, TIM-4, 4-IBB, BTLA, CD39, CD73 and A2aR, and, if applicable, cognate binding partners (e.g. PD-1 and PD-L1), (see for example, FIG. 1 and Table 1 in Marin-Acevedo et al. (2018) J Hematol Oncol. 15; 11(1):39. doi: 10.1186/s13045-018-0582-8).

As used herein, “immune checkpoint modulators” include immune checkpoint agonists and immune checkpoint antagonists. In particular the immune checkpoint modulator is an (direct) inhibitor (antagonist) of an inhibitory immune checkpoint (e.g. an inhibitor of PD-L1/PD-1) or an (direct) stimulator (agonist) of an stimulatory immune checkpoint (e.g. an agonist of OX40), including the immune checkpoint proteins and pathways mentioned above, and, if applicable, its cognate binding partners. In general immune checkpoint modulators as used herein refers to a compound(s) or pharmaceutical agent(s) or drug(s) or candidate drug(s) (e.g. antibodies, fusion proteins, small molecule drugs (natural or synthetic), interfering RNA (e.g. siRNA) that totally or partially modulate (e.g. interferes with one or more immune checkpoints or their ligands, in particular inhibitory immune checkpoint molecules such as PD-1 or CTLA-4 and/or the PD-1 ligand PD-L1).

Non-limiting examples of PD-1 inhibitor compounds include PD-1 antibodies such as nivolumab (Opdivo®, Bristol-Myers Squibb), pembrolizumab (Keytruda®, Merck), BGB-A317, and others such as PDR001/spartalizumab (Novartis). Further PD-1 inhibitors also include any anti-PD-1 antibody described in U.S. Pat. Nos. 8,008,449, 7,521,051 and 8,354,509. Also contemplated are fusion proteins that bind to PD-1 (e.g. anti-PD-1 fusion proteins AMP-224 (Medlmmune) and AMP-514 (Medlmmune)). Non-limiting examples of PD-L1 inhibitor compounds include anti-PD-L1 antibodies such as durvalumab (MEDI4736, Imfinzi®, Medlmmune), atezolizumab (Tecentriq®, Roche), avelumab (Bavencio®, Merck), and others such as BMS-936559 (BMS) (Meng et al (2015), Cancer Treatment Review, Vol. 41, pages 868-876; Brahmer et al (2010) J Clin Oncol 28:3167-75; Brahmer et al (2012) N. Engl. J. Med. Vol: 366, pages 2455-65; Flies et al (2011) Yale J. Biol. Med. Vol. 84, pages 409-21; Topalian et al. (2012b) N. Engl. J. Med. Vol. 366, pages 2443-54; Diggs et al (2017), Biomarker Research, Vol. 5:12, pages 1-6). Further PD-L1 inhibitors include any anti-PD-L1 antibody described in US. Pat. No. 8,383,796. Also contemplated are fusion proteins that bind to PD-L1.

Non-limiting examples of CTLA-4 inhibitor compounds include ipilimumab ((Yervoy®, MDX-010, Bristol-Myers Squibb, FDA approved for melanoma in 2011) and (not yet approved) is tremelimumab (CP-675206, Pfizer) (Postow et al (2015) J. Clinical oncology, Vol. 33, pages 1974-1983; Pardoll, D. et al (2012), Nature Reviews Cancer, Vol. 12, pages 252-264).

Currently clinical approved checkpoint inhibitors block CTLA-4 and PD-1, and also one of the ligands of PD-1, PD-L1. In the context of the present invention modulators, e.g. inhibitors of PD-1 and PD-L1, are particularly preferred, alone or in combination with other immune checkpoint therapy agents such as CTLA-4 modulators (e.g. inhibitors). Various clinical trials are under way with immune checkpoint modulators in many different types of cancer (see, e.g. Darvin et al (2018) Exp Mol Med. 50(12): 165., doi: 10.1038/s12276-018-0191-1), and next-generation immune checkpoint modulators are under development (see, e.g. Marin-Acevedo et al. (2018) J Hematol Oncol. 15;11(1):39. doi: 10.1186/s13045-018-0582-8.), including drugs blocking LAG-3, TIM-3, TIGIT, VISTA, or B7/H and agonists of stimulatory checkpoint pathways such as OX40, ICOS, GITR, 4-1BB, CD40.

Preferred immune checkpoint modulators for use in the treatment of cancer according to the invention are modulators of PD-1, PD-L1, CTLA-4, LAG-3, and CD47.

Preferred immune checkpoint modulators for use in the treatment according to the inventions are ipilimumab, pembrolizumab, nivolumab, spartalizumab, durvalumab, and atezolizumab

Also provided is for Staphylococcal enterotoxin for use in the treatment of cancer according to the invention wherein administration of the Staphylococcal enterotoxin is intratumoral and/or adjacent to the tumor and/or wherein administration of the immune checkpoint modulator is systemic, intratumoral and/or adjacent to the tumor.

Although the route of administration of the Staphylococcal enterotoxin to the subject is not in any particular way limited, in a preferred embodiment, the

Staphylococcal enterotoxin in provided intratumoral and/or adjacent to the tumor. In other words, Staphylococcal enterotoxin may, according to the current invention, be administered to the subject via any suitable route of administration, including but not limited to, parenteral, intravenous, intra-arterial, intramuscular, intratumoral and oral routes of administration, and in any form and suitable formulation.

However, in a preferred embodiment the Staphylococcal enterotoxin is administered to the patient at least partially within the tumor (intratumoral) or adjacent to the tumor. The skilled person is aware of available techniques and formulation requirements with respect to the intratumoral administration of drugs, including Staphylococcal enterotoxins and/or with respect to the administration of a drug adjacent to the tumor. According to the invention it is not required that all Staphylococcal enterotoxin is administered intratumoral, as long as at least part is provided intratumoral, or, alternatively, provided adjacent to the tumor. The term adjacent, within the context of the current invention relates to administration of the drug to tissue directly surrounding the tumor such that the drug can diffuse/be transported into the tumor in high concentrations. For example, the drug may be injected in tissue just below or on top of the tumor in the subject. Drugs, therapeutic agents, medicaments and compositions may be formulated in fluid or solid form. Fluid formulations may be formulated for administration by injection to a selected region of the human or animal body.

Within the context of the invention, the immune checkpoint modulator may be administered to the subject using any suitable and common route of administration. Current available immune checkpoint modulators are commonly provided systemically (i.e. via the circulation) and the skilled person is aware of available techniques and formulation requirements with respect to the systemic administration of drugs, including immune checkpoint modulators. In the context of the current invention, in an embodiment, the immune checkpoint inhibitors are preferably provided systemically. However, according to another embodiment, the immune checkpoint modulators are preferably provided intratumoral and/or adjacent to the tumor, and as already discussed herein with respect to the administration of Staphylococcal enterotoxin.

The skilled person knows how, within the context of the current invention, determine therapeutically effective amounts of Staphylococcal enterotoxin and/or of the immune checkpoint modulator. Such effective amounts may be determined routinely, based on pharmacological effectiveness as well as on physiological safety (toxicity) in the context of the treatment according to the invention.

Also provided is for Staphylococcal enterotoxin for use in the treatment of cancer according to the invention wherein the Staphylococcal enterotoxin is selected from the group consisting of Staphylococcal enterotoxin A, B, C, D, E, F, G, I, M, N, O, Q and U, or biologically active fragments thereof, preferably wherein the

Staphylococcal enterotoxin is Staphylococcal enterotoxin B.

Although the current invention is not in particular limited to any particular Staphylococcal enterotoxin, in a preferred embodiment, the Staphylococcal enterotoxin is selected from the group consisting of Staphylococcal enterotoxin A, B, C (including C1, C2 and C3), D, E, F, G, I, M, N, O, Q and U (SEA, SEB, SEC, SED,

SEE, SEF, SEG, SEI, SEM, SEN, SEO, SEQ, and SEU) or biologically active fragments thereof. These different Staphylococcal enterotoxins are known to the skilled person, and amino acid sequence, and nucleotide sequences of SEA, SEB, SEC, SED, SEE, SEF, SEG, SEI, SEM, SEN, SEO, SEQ, and SEU are available in the art (see e.g. the following Uniprot identifiers (www.uniprot.org): SEA: P0A0L2; SEB: A0A0H2WZB2; SEC1: P01553; SEC2: P34071; SEC3: P0A0L5; SED: P20723; SEE: P12993; SEG: P0A0L8; SEI: Q8RR75; SEM: A0A0H3K005; SEN A0A0H3JQM6; SEO A0A0H3JQN2; SEQ: A0A0H3JX85 and SEU: A0A5F0HMJ4).

The skilled person knows how to prepare and formulate such Staphylococcal enterotoxins for use in the treatment according to the invention.

In a particular preferred embodiment, the Staphylococcal enterotoxin is Staphylococcal enterotoxin B. Staphylococcal enterotoxin B has been described in detail in the prior art, including the existence of Staphylococcal enterotoxin B variants isolated from different S. aureus isolates (Kohler et al (2012) PLoS One. 7(7): e41157, doi: 10.1371/journal.pone.0041157) and amino acid sequence, 3D structure and biological active fragments are known to the skilled person.

Staphylococcal enterotoxins, including. For example, A, B and C, are commercially available, from, e.g. Merck/Sigmaaldrich and others. Although, preferably one type of Staphylococcal enterotoxin is used in a formulation for use in the treatment according to the invention, it is contemplated that more than one different types of Staphylococcal enterotoxin may be used in the treatment. Alternatively, different fragment of one type of Staphylococcal enterotoxin may be used, for example, different biological active fragments from Staphylococcal enterotoxin B. In the context of the current invention, “biological active fragments” relate to fragment from the Staphylococcal enterotoxin that are able to improve/enhance T cell infiltration is a tumor, for example in accordance with the examples as described herein.

Accordingly, there is also provided for Staphylococcal enterotoxin for use in the treatment of cancer according to the invention wherein the Staphylococcal enterotoxin is a biologically active fragment of an Staphylococcal enterotoxin.

Based on the disclosure herein, the skilled person understands how to determine if a particular fragment of an Staphylococcal enterotoxin is biologically active. For example, the skilled person may provide biological active fragments of Staphylococcal enterotoxin by removing or replacing one or more amino acids of the Staphylococcal enterotoxin, or by adding one or more amino acids. Alternatively, fragments of the Staphylococcal enterotoxin can be provided, for example, by treatment of the protein with a protease, or by providing a vector only expressing part of the Staphylococcal enterotoxin. Also contemplated are fusion protein comprising Staphylococcal enterotoxin, or biologically active fragments thereof, fused to other peptides or compounds./pct

Also provided is for Staphylococcal enterotoxin for use in the treatment of cancer according to the invention wherein the immune checkpoint modulator is selected from the group consisting of a immune checkpoint modulator targeting PD-1, PD-L1, PD-L2, CTLA-4, LAG3, B7-H3, B7-H4, B7-H5 (VISTA), KIR, TIGIT, CD47, TIM3, CD70, CD27, CD160, CD112, CD134 (OX40), CD226, CD155, A2aR, CD40, CD40L, CD137, CD270, CD272, CD275, CD278, CD28, GITR (TNFS18), B7-H2, B7-H6, HVEM, B7.1, TIM-1, TIM-4, 4-IBB, BTLA, CD39, CD73 and A2aR, and, if applicable, cognate binding partners (e.g. PD-1 and PD-L1), preferably wherein the immune checkpoint modulator is an immune checkpoint inhibitor, preferably an immune checkpoint inhibitor selected from the group consisting of a LAG-3, CD47, CTLA-4, PDL-1 or PD-1 immune checkpoint inhibitor.

As discussed herein, in recent years several immune checkpoint have been identified, and which are al considered as targets in immunotherapy, in particular in immune checkpoint therapy. In addition to inhibitory immune checkpoints also stimulatory immune checkpoints have been identified. It will be understood by the skilled person that the current invention now allows the treatment of tumors that do not respond, or that do no longer respond, or that only respond to a limited degree to interventions directed to immune checkpoints (bot stimulatory and inhibitory). By including, as part of the treatment, the use of an Staphylococcal enterotoxin, targeting immune checkpoints either becomes available as a treatment strategy or becomes more efficient compared to treatments that do not include the use of an Staphylococcal enterotoxin. Therefor the skilled person knows that an Staphylococcal enterotoxin may be used in combination with any kind of immune checkpoint modulator directing any kind of immune checkpoint.

However, in a preferred embodiment the immune checkpoint modulator is directed to PD-1, PD-L1, CTLA-4, LAG-3, and/or CD47.

As disclosed herein, the immune checkpoint are all well-known to the skilled person and described and detailed in a wide range of scientific articles.

In a preferred embodiment the immune checkpoint modulator is an immune checkpoint agonist or stimulator, even more preferably the immune checkpoint modulator is an agonist or stimulator of a stimulatory immune checkpoint, for example of those disclosed herein.

In a preferred embodiment the immune checkpoint modulator is an immune checkpoint antagonist or inhibitor, even more preferably the immune checkpoint modulator is an antagonist or inhibitor of an inhibitory immune checkpoint, for example of those disclosed herein, preferably the immune checkpoint inhibitor is an inhibitor of LAG-3, CD47, CTLA-4, PDL-1 or PD-1.

Also provided is for Staphylococcal enterotoxin for use in the treatment of cancer according to the invention wherein the tumor is a solid tumor, a cold or low immune cell infiltrated tumor, a immunotherapy resistant tumor, a primary tumor, and/or a secondary tumor.

In a preferred embodiment, the tumor is a solid tumor. Solid tumors are abnormal mass of tissue that usually does not contain cysts or liquid areas. Solid tumors may be benign (not cancer), or malignant (cancer). Thus in an embodiment the tumor in the subject having cancer is a benign tumor. In a preferred embodiment, the tumor is a cold tumor, i.e. tumors that lack of or has low T cell infiltration (Galon et al. (2019) Nature Reviews Drug Discovery 18: 197-218, doi:10.1038/s41573-018-000 and Bonaventura et al. (2019) Front. Immunol., doi.org/10.3389/fimmu.2019.00168). The skilled person knows the term “cold tumor”.

As disclosed herein, current immune checkpoint-modulating agents have demonstrated clinical efficacy in only certain tumor types. These responsive tumors have, in general a high burden of tumor-specific neoantigens, high tumor-mutational burden, and abundant tumor-infiltrating T cells. But also these tumors often stop responding, which may be due to T cell exhaustion, decreased T cell effector function, and/or upregulated inhibitory checkpoints. As disclosed herein, the method according to the invention can suitable be used to “re-activate” the anti-cancer immune response in these types of tumors.

In contrast, tumors with low tumor-mutational burden, low neoantigen burden, and/or a paucity of T cells are referred to as immunologically “cold,”. These types of tumors first require the addition of agents to facilitate the induction of T cells into tumors. Cold tumors also often recruit immunosuppressive cell subsets, including regulatory T cells, myeloid-derived suppressor cells, and macrophages, and secrete immunosuppressive soluble cytokines, chemokines, and metabolites. In view of the data disclosed herein, therefore, in a preferred embodiment of the invention, the subject having cancer has a tumor that would be referred to by the skilled person as a “cold tumor”.

In a preferred embodiment, the tumor is an immunotherapy resistant, preferably immune checkpoint modulator resistant, tumor.

In a preferred embodiment, the tumor is a primary tumor. In another preferred embodiment, the tumor is a secondary tumor.

In a preferred embodiment, the cancer/tumor is colon cancer, pancreatic cancer, breast cancer, ovarian cancer, prostate cancer, lung cancer, head-neck cancer, thyroid cancer, skin cancer, including melanoma, hepatic cancer, or bladder cancer.

Also provided is for Staphylococcal enterotoxin for use in the treatment of cancer according to the invention wherein the tumor is characterized by
(a) a low interferon gamma response signature or a low interferon gamma response expanded signature;
(b) a low BATF3 signature; and/or
(c) a low Tumor Infiltration Signature.

A tumor with a low interferon (IFN) gamma response signature is herein defined as a tumor comprising cells with a mutation in and/or with reduced expression of at least one gene, preferably all genes, selected from the IFN gamma gene-signature described by Ayers et al. (2017) J Clin. Invest.; 127(8):2930-2940, doi: 10.1172/JCI91190) which gene-signature consists of the genes: ID01, CXCL10, CXCL9, HLA-DRA, STAT1 and IFNG.

Preferably, a low IFN gamma response signature is defined as the expression of the (Ayers) IFN gamma response (gene) signature observed in the lowest 50%, 60%, 70%, 73%, 75%, 80%, 85%, 89%, 90% or 95% of patients. More preferably, a low IFN gamma response signature is defined as the expression of the (Ayers) IFN gamma response (gene) signature observed in the lowest 81%, or the 81st percentile, of patients.

A tumor with a low interferon (IFN) gamma response expanded signature is herein defined as a tumor comprising cells with a mutation in and/or with reduced expression of at least one gene selected from the interferon (IFN) gamma response expanded (gene) signature described by Ayers et al. (2017) J Clin Invest.; 127(8):2930-2940, doi: 10.1172/JCI91190) which gene-signature consists of the genes CD3D, IL2RG, IDO1, NKG7, CIITA, HLA-E, CD3E, CXCR6, CCL5, LAG3, GZMK, TAGAP, CD2, CXCL10, HLA-DRA, STAT1, CXCL13 and GZMB.

Preferably, a low IFN gamma response expanded signature is defined as the expression of the (Ayers) IFN gamma response expanded (gene)-signature observed in the lowest 50%, 60%, 70%, 73%, 75%, 80%, 85%, 89%, 90% or 95% of patients. More preferably, a low IFN gamma response signature is defined as the expression of the (Ayers) IFN gamma response expanded (gene)-signature observed in the lowest 81%, or the 81st percentile, of patients.

For melanoma, preferably the low interferon (IFN) gamma response expanded signature is used; for other cancers preferably the low interferon (IFN) gamma response signature is used.

A tumor with a low BATF3 signature is herein defined as a tumor comprising cells with a mutation in and/or with reduced expression of at least one gene selected from the BATF3 gene-signature described by Liu et al (2019) ONCOIMMUNOLOGY 8 (2), e1546068, doi: 10.1080/2162402X.2018.1546068) which gene signature consists of the genes: XCR1, IRF8, CLEC9A, BATF3, and THBD.

Preferably, a low BATF3 signature is defined as the expression of the (Liu) BATF3gene-signature observed in the lowest 50%, 60%, 70%, 73%, 75%, 80%, 85%, 89%, 90% or 95% of patients. More preferably, a low BATF3 signature is defined as the expression of the (Liu) BATF3 gene-signature observed in the lowest 81%, or the 81st percentile, of patients.

A tumor with a low Tumor Infiltration Signature is herein defined as a tumor comprising cells with a mutation in and/or with reduced expression of at least one gene selected from the Tumor Infiltration Signature gene-signature described by Danaher et al (2018) Journal for ImmunoTherapy of Cancer 6:63, doi: 10.1186/s40425-018-0367-1) which gene signature consists of the genes: PSMB10, HLA-DQA1, HLA-DRB1, CMKLR1, HLA-E, NKG7, CD8A, CCLS, CXCL9, CD27, CXCR6, IDO1, STAT1, TIGIT, LAG3, CD274, PDCD1LG2, and CD276.

Preferably, a low Tumor Infiltration Signature is defined as the expression of the (Danaher) Tumor Infiltration Signature gene-signature observed in the lowest 50%, 60%, 70%, 73%, 75%, 80%, 85%, 89%, 90% or 95% of patients. More preferably, a low Tumor Infiltration Signature is defined as the expression of the (Danaher) Tumor Infiltration gene-signature observed in the lowest 81%, or the 81st percentile, of patients.

The expression levels of the signature genes can be determined using methods known in the art per se such as RT-qPCR, RNA sequencing or Nanostring analysis (NanoString Technologies, Inc.). Preferably, the expression level signature genes is determined in vitro in a sample of the tumor, e.g. obtained in a biopsy from the patient.

The tumor may be characterized by a low interferon gamma response signature or a low BATF3 signature or a low Tumor Infiltration Signature. In a preferred embodiment, the tumor is characterized by at least two, preferably at least three of a low interferon gamma response signature, a low BATF3 signature and a low Tumor Infiltration Signature.

Also provided is for Staphylococcal enterotoxin for use in the treatment of cancer according to the invention wherein administration of the Staphylococcal enterotoxin and administration of the immune checkpoint modulator is simultaneous, separate, or sequential.

According to the invention, the Staphylococcal enterotoxin and the immune checkpoint modulator may be provided in the same pharmaceutical formulation, or may be provided in separate pharmaceutical formulation. The Staphylococcal enterotoxin and the immune checkpoint modulator may be administered simultaneously, i.e. at the same time, separate from each other or sequential from each other. The Staphylococcal enterotoxin and the immune checkpoint modulator may be administered according to different dosing schedules or according to the same dosing schedules. For example the Staphylococcal enterotoxin and/or the immune checkpoint modulator may be administered one daily, or more times daily, and/or once every one, two, three, for, seven days, or more, weekly, every two weeks, every three weeks or more. The Staphylococcal enterotoxin may be administered more often than the immune checkpoint modulator or the immune check modulator may be administered more often than the Staphylococcal enterotoxin and so on.

Also provided is for Staphylococcal enterotoxin for use in the treatment of cancer according to the invention, wherein administration of the Staphylococcal enterotoxin and/or administration of the immune checkpoint modulator is by injection.

The skilled person knows of the prepare formulations suitable for injection and how to inject such formulation, for example, intratumoral.

Also provided is for Staphylococcal enterotoxin for use in the treatment of cancer according to any of the previous claims wherein the treatment comprises administration of more than one Staphylococcal enterotoxin and/or more than one immune checkpoint modulator and/or wherein the treatment further comprises the administration of one or more further drugs. Such further drugs or agent may be a low molecular weight compound, e.g. a small molecule, but may also be a larger compound, for example, an oligomer of nucleic acids, amino acids, or carbohydrates including without limitation proteins, oligonucleotides, ribozymes, DNAzymes, glycoproteins, siRNAs, lipoproteins, aptamers, and modifications and combinations thereof. The drug or agent may be a cancer drug, but may also be directed to other conditions or be directed to side-effects associated with the cancer in the subject.

In certain embodiments, the further drug or agent is selected from the group consisting of tetrabenazine, dihydrotetrabenazine, ketamine, pirfenidone, phenylephrine, ethambutol, venlafaxine, zolipidem, esomeprazole, lansoprazole, omeprazole, pantoprazole, rabeprazole, sitaxentan, codeine, hydrocodone, morphine, oxycodone, almotriptan, eletriptan, naratriptan, sumatriptan, zolmitriptan, ranolazine, desmethylvenlafaxine, mirabegron, ticagrelor, darapladib, rilapladib, nilotinib, tofacitinib, apixaban, lumiracoxib, solabegron, riociguat, cariprazine, neratinib, pelitinib, fostamatinib, R-406, dihydrotetrabenazine, NBI-98854, nintedanib, F-351, agomelatine, almorexant, alogliptin, anastrozole, aripiprazole, atomoxetine, bosentan, brivaracetam, bupropion, cediranib, cinacalcet, clemizole, dextromethorphan, dimeboline, donepezil, duloxetine, fingolimod, gefitinib, imatinib, ITMN-191, ivabradine, linezolid, lonafarnib, maraviroc, mosapride, nateglinide, oxybutynin, paroxetine, pazopanib, quetiapine, rilpivirine, rimonabant, rolofylline, sitagliptin, tolterodine, udenafil, valproic acid, vandetanib, vildagliptin, alpha-lipoic acid, ambrisentan, anacetrapib, apremilast, atazanavir, bardoxolone, baricitinib, boceprevir, brecanavir, carfilzomib, carmofur, cilostazol, conivaptan, crizotinib, darunavir, dasatinib, dimethylcurcumin, dolutegravir, elvitegravir, erlotinib, etravirine, felbamate, filibuvir, gliclazide, ibudilast, ibrutinib, idebenone, iloperidone, iloprost, indiplon, ivacaftor, L-838417, lacosamide, lapatinib, lenalidomide, lorcaserin, mibefradil, milnacipran, N-butyl bumetanide, NTP-2014, niacin, niacin prodrugs, NS11394, NS-304, MRE-304, MRE-269, pagoclone, pentifylline, pentoxifylline, pentoxifylline metabolites, PLX4032, pomalidomide, ponatinib, PPL-100, praziquantel, preladenant, primaquine, radequinil, raltegravir, rigosertib, rivaroxaban, ruxolitinib, safinamide, silodosin, sodium oxybate, 4-hydroxybutyrate, sorafenib, telcagepant, thalidomide, tigecycline, omadacycline, tizanidine, TPA-023, treprostinil, varespladib, vercimon, vicriviroc, levodopa, carbidopa, levodopa in combination with carbidopa, amantadine, dipraglurant, nintedanib, and pridopidine

According to another aspect also provided is for an immune checkpoint modulator for use in the treatment of cancer in a subject having a tumor, wherein the treatment further comprises administration of a Staphylococcal enterotoxin.

As will be understood by the skilled person, all embodiments, preferences and explanation provided above aspect of the invention can be implemented with respect to any further aspect of the invention described herein.

According to another aspect also provided is for Staphylococcal enterotoxin in combination with an immune checkpoint modulator for use in the treatment of cancer in a subject having a tumor.

As will be understood by the skilled person, all embodiments, preferences and explanation provided above aspect of the invention can be implemented with respect to any further aspect of the invention described herein.

According to another aspect also provided is for a pharmaceutical combination comprising a Staphylococcal enterotoxin and an immune checkpoint modulator for use in the treatment of cancer in a subject having a tumor.

As will be understood by the skilled person, all embodiments, preferences and explanation provided above aspect of the invention can be implemented with respect to any further aspect of the invention described herein.

According to another aspect also provided is for a method for the treatment of cancer in a subject having a tumor, wherein the method comprises administration of an effective amount if a Staphylococcal enterotoxin and administration of an effective amount of an immune checkpoint modulator to the subject. As will be understood by the skilled person, all embodiments, preferences and explanation provided above aspect of the invention can be implemented with respect to any further aspect of the invention described herein.

According to another aspect also provided is for a method for enhancing therapeutic efficacy of an immune checkpoint modulator in a subject, wherein the method comprises the administration of a Staphylococcal enterotoxin and the administration of an immune checkpoint inhibitor to the subject.

As will be understood by the skilled person, all embodiments, preferences and explanation provided above aspect of the invention can be implemented with respect to any further aspect of the invention described herein.

It will be understood that all details, embodiments and preferences discussed with respect to one aspect of embodiment of the invention is likewise applicable to any other aspect or embodiment of the invention and that there is therefore not need to detail all such details, embodiments and preferences for all aspect separately.

Having now generally described the invention, the same will be more readily understood through reference to the following examples which is provided by way of illustration and is not intended to be limiting of the present invention.

### EXAMPLES

Introduction

Immune based therapies brought about a revolution in oncology. These therapies were shown to be effective in multiple cancers such as melanoma, lung cancer an renal cancer. Yet, not all patients respond to immune based therapies.

These patients can be identified by low frequencies of tumor infiltrating immune cells, low BATF3 signature and/or low IFNγ signature(s) and the tumors are commonly referred to as “cold” tumors. It is for these patients that we are in need of novel therapeutic strategies.

The strategy disclosed herein is aimed at converting the “cold” tumors into hot tumors. It data unexpectedly show that this can be achieved by means of bacterial superantigens such as Staphylococcal enterotoxins. The data show that exposure of immune cells to superantigens result in hyperactivation, and increased T cell infiltration in tumors, including cold tumors. The data also suggest that the treatment according to the invention may be used to improve immune checkpoint modulator treatment independent on whether the tumor is “hot” or “cold”.

Disclosed herein are results with the superantigen Staphylococcal enterotoxin B (SEB), although other superantigens show comparable results. As a model for “cold” tumors we used our inducible Tyr::CreER^{T2}; Pten^{LoxP/LoxP}; Braf^{CA/+} melanoma model (see Hooijkaas, A. I., et al., Targeting BRAFV600E in an inducible murine model of melanoma. Am J Pathol, 2012. 181(3): p. 785-94.) These tumors are characterized by low immune infiltration (FIG. 1b) and lack of response to immune based therapies.

First we performed an experiment to test the effect of SEB on immune infiltration (FIG. 1a). We used a mixture of tumors that spontaneously developed on Tyr::CreER^{T2}; Pten^{LoxP/LoxP}; Bref^{CA/+} mice and tumors that were induced with 4-OH tamoxifen in Tyr::CreER^{T2}; Pten^{LoxP/LoxP}; Bref^{CA/+} mice. Tumors were injected with vehicle or with 50 μg SEB when tumors developed growth in 3 dimensions (100-1000 mm³, size determined by caliper). A maximum volume of 50 ul was administered to the base of the tumor divided over multiple injections. Tumor sizes in both groups were matched. Mice were sacrificed by cervical dislocation two days after treatment, and tumors were fixed in formalin and embedded in paraffin. Slides were stained for CD3 by our animal pathology department and scored qualitatively by the researcher.

Infiltration was scored in three categories: “absent”, “low” and “high” (FIG. 1b). Intratumoral injection of SEB led to “high” T cell infiltration in multiple tumors, while high infiltration was never observed in vehicle injected tumors (FIG. 1c).

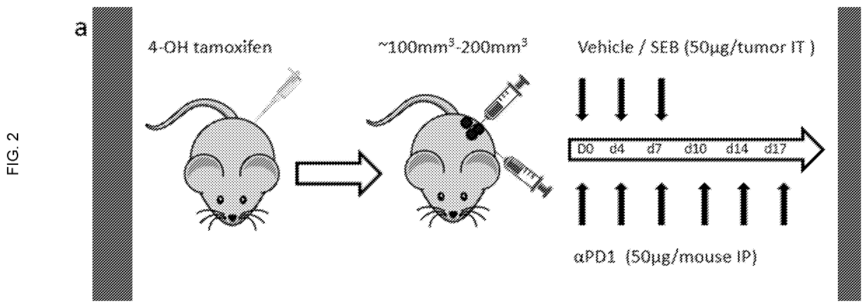

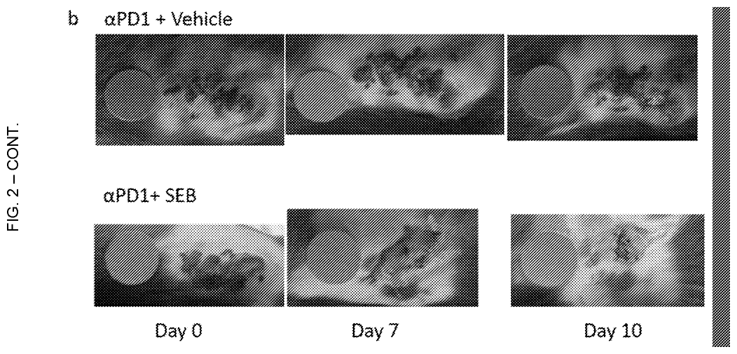

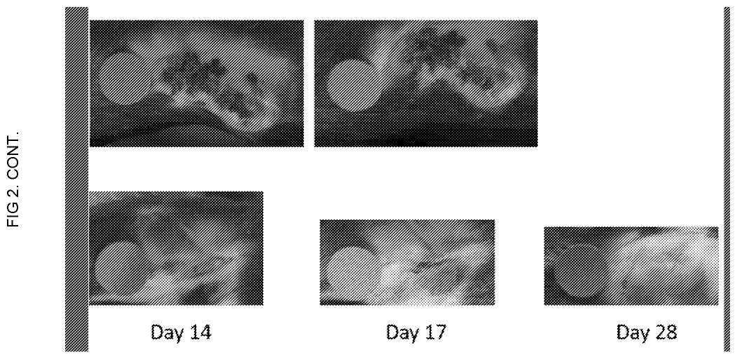

After we established increased T cell infiltration in our model of “cold” melanoma's by SEB we combine superantigen with immune checkpoint blockade (FIG. 2a). Tumors were induced on the skin as above. Tumor growth was followed twice weekly (size determined by analysis of pictures taken with a reference) until humane endpoints were reached in accordance with the guidelines of our animal facility. Mice were divided over two groups when tumors reached 100-200 mm². One group received two times weekly intratumoral injections of vehicle plus intraperitoneal injections of anti-PD1 (αPD1, clone RMP1-14; 100 μg) and the other group received two times weekly intratumoral injections of SEB (50 μg) plus intraperitoneal injections of the same anti-PD1 (100 μg). SEB treatment was ceased after three injections and lethal toxicity was not observed. Injections of αPD1was continued for a total of 4 weeks. The melanotic part of the SEB injected tumors disappeared completely, followed by a reduced outgrowth speed of the amelanotic part (FIG. 2b). Of note, depigmentation of the fur around SEB injected tumors was observed. Amelanotic tumors that did not receive SEB injection present on a mouse with a tumor that received SEB appear to grow unaffected. Growth of these non-injected tumors was the reason that mice in this experiment needed to be sacrificed as the treated tumor developed slowly.

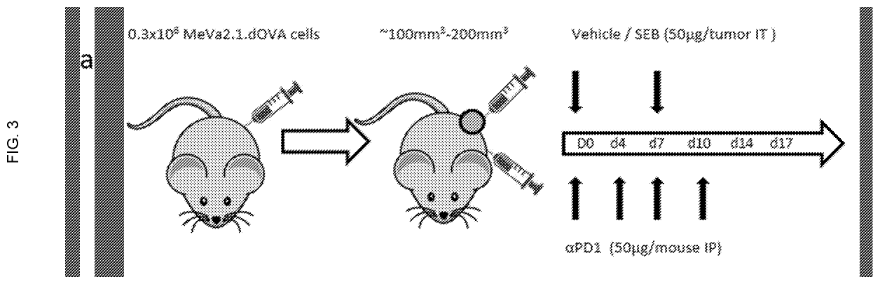

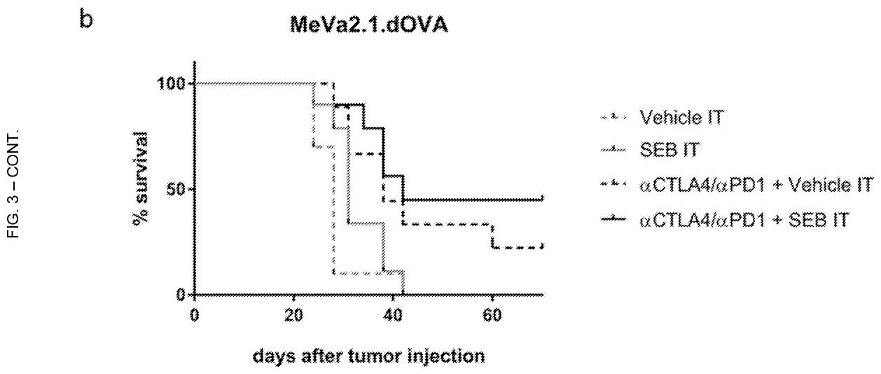

Prompted by this remarkable results in our “cold” melanoma model we decided to test the combination of and antigen such as SEB and immune checkpoint blockade in a transplantable immune refractory model: MeVa2.1.dOVA. MeVa2.1.dOVA cells express the intracellular domain of OVA(dOVA) which is strongly antigenic. Yet, MeVa2.1.dOVA tumors grow identical in the presence or absence of a functioning immune system, (data not shown).However, the combination of twice weekly αCTLA4 (clone 9D9; 50 μg) plus αPD1 (clone RMP1-14; 100 μg) increases tumor control (FIG. 3a). In this experiment we decided to lower the dose of SEB (50 μg) from twice weekly to one time a week. C57BL/6 mice were inoculated with 0.3×10⁶ cells at the start of the experiment. Tumor size was followed with a caliper twice weekly for the entire duration of the experiment. When tumors reached ˜200 mm³ mice were randomized in 4 groups based on tumor size. Mice either received weekly intratumoral injection with vehicle or SEB (50 μg), these two groups were further divided in groups that received twice weekly αCTLA4 (50 μg) plus αPD1 (100 μg) or nothing. In this experiment lethal toxicity was observed in 3/20 mice treated with SEB. Again depigmentation of the fur around SEB injected tumors was observed. The amount of mice that rejected the tumor doubled upon SEB treatment (FIG. 3b).

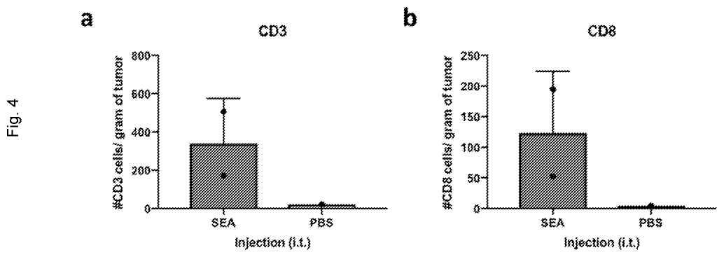

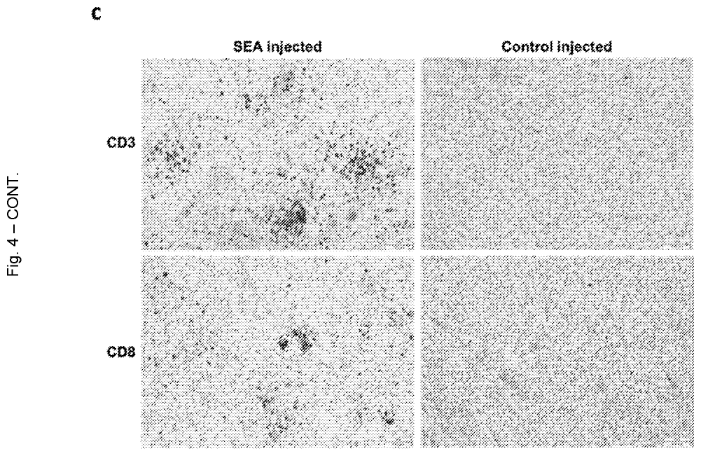

In order to show that the effects are not limited to SEB but can also be obtained by other Staphylococcal enterotoxins, the experiment with the TyrCreERT2; PtnF; BRAFV600E mice described above was repeated with Staphylococcal enterotoxin A (SEA). Tumors (spontaneous or tamoxifen induced) from TyrCreERT2;PtnF; BRAFV600E mice show very low immune infiltration and in this mouse model, we tested if intratumor (i.t.) administration of staphylococcal enterotoxin A (SEA) also leads to rapid T cell infiltration. Tumor bearing mice were injected (i.t) with 50 microgram SEA (n=2) or vehicle control (PBS, n=1). About 48 hours after injection, tumors were harvested to assess T cell infiltration, using immunohistochemistry (IHC) and flow cytometry. We observed that tumor injected with SEA had a remarkable increase in number of CD3+ T cells and CD3+CD8+ T cells (FIG. 4a, b). This finding was further supported by IHC analysis, in which tumor injected with SEA showed a high CD3+ T cell and CD8+ T cell infiltration throughout the tumor, compared with mice injected with vehicle control (FIG. 4c). This data supports the finding that not only staphylococcal enterotoxin B (SEB), but also injection of SEA leads to an inflammation and immune infiltration.

The above results show that tumors that lack responsive to cancer immunotherapy, or only show limited response, can successfully be treated by treatment of the tumors with superantigens such as SEB, together with, i.e. in combination with (existing) cancer immunotherapy Adding treatment with superantigens to cancer immunotherapy broadens the type and number of tumors that respond to cancer immunotherapy and/or further enhance the efficacy of cancer immunotherapy of existing approaches.

Having now fully described this invention, it will be appreciated by those skilled in the art that the same can be performed within a wide range of equivalent parameters, concentrations, and conditions without departing from the spirit and scope of the invention and without undue experimentation.

While this invention has been described in connection with specific embodiments thereof, it will be understood that it is capable of further modifications. This application is intended to cover any variations, uses, or adaptations of the inventions following, in general, the principles of the invention and including such departures from the present disclosure as come within known or customary practice within the art to which the invention pertains and as may be applied to the essential features hereinbefore set forth as follows in the scope of the appended claims.

All references cited herein, including journal articles or abstracts, published or corresponding patent applications, patents, or any other references, are entirely incorporated by reference herein, including all data, tables, figures, and text presented in the cited references. Additionally, the entire contents of the references cited within the references cited herein are also entirely incorporated by references.

Reference to known method steps, conventional methods steps, known methods or conventional methods is not in any way an admission that any aspect, description or embodiment of the present invention is disclosed, taught or suggested in the relevant art.

The foregoing description of the specific embodiments will so fully reveal the general nature of the invention that others can, by applying knowledge within the skill of the art (including the contents of the references cited herein), readily modify and/or adapt for various applications such specific embodiments, without undue experimentation, without departing from the general concept of the present invention. Therefore, such adaptations and modifications are intended to be within the meaning and range of equivalents of the disclosed embodiments, based on the teaching and guidance presented herein. It is to be understood that the phraseology or terminology herein is for the purpose of description and not of limitation, such that the terminology or phraseology of the present specification is to be interpreted by the skilled artisan in light of the teachings and guidance presented herein, in combination with the knowledge of one of ordinary skill in the art.

1. A method for treating cancer in a subject having a tumor, comprising administering to the subject
Staphylococcal enterotoxin, or a biological active fragment thereof, and
an immune checkpoint modulator.

2. The method of claim 1, wherein administration of the Staphylococcal enterotoxin is intratumoral and/or adjacent to the tumor and/or wherein administration of the immune checkpoint modulator is systemic, intratumoral and/or adjacent to the tumor.

3. The method of claim 1, wherein the Staphylococcal enterotoxin is selected from the group consisting of Staphylococcal enterotoxin A, B, C, D, E, F, G, I, M, N, O, Q and U, preferably wherein the Staphylococcal enterotoxin is Staphylococcal enterotoxin B.

4. The method of claim 1, wherein the Staphylococcal enterotoxin is a biologically active fragment of an Staphylococcal enterotoxin.

5. The method of claim 1, wherein the immune checkpoint modulator is selected from the group consisting of a immune checkpoint modulator targeting PD-1, PD-L1, PD-L2, CTLA-4, LAG3, B7-H3, B7-H4, B7-H5 (VISTA), KIR, TIGIT, CD47, TIM3, CD70, CD27, CD160, CD112, CD134 (OX40), CD226, CD155, A2aR, CD40, CD40L, CD137, CD270, CD272, CD275, CD278, CD28, GITR (TNFS18), B7-H2, B7-H6, HVEM, B7.1, TIM-1, TIM-4, 4-IBB, BTLA, CD39, CD73 and A2aR, preferably wherein the immune checkpoint modulator is an immune checkpoint inhibitor, preferably an immune checkpoint inhibitor selected from the group consisting of a LAG-3, CD47, CTLA-4, PDL-1 or PD-1 immune checkpoint inhibitor.

6. The method of claim 1, wherein the tumor is a solid tumor, a cold or low immune cell infiltrated tumor, a immunotherapy resistant tumor, a primary tumor, and/or a secondary tumor.

7. The method of claim 1, wherein the tumor is characterized by
(a) a low interferon gamma response signature or a low interferon gamma response expanded signature;
(b) a low BATF3 response signature; and/or
(c) a low Tumor Infiltration Signature.

8. The method of claim 1, wherein administration of the Staphylococcal enterotoxin and administration of the immune checkpoint modulator is simultaneous, separate, or sequential.

9. The method of claim 1, wherein administration of the Staphylococcal enterotoxin and/or administration of the immune checkpoint modulator is by injection.

10. The method of claim 1, wherein the treatment comprises administration of more than one Staphylococcal enterotoxin and/or more than one immune checkpoint modulator and/or wherein the treatment further comprises the administration of one or more further drugs.

11. (canceled)

12. (canceled)

13. A pharmaceutical composition comprising a combination of a Staphylococcal enterotoxin and an immune checkpoint modulator, wherein the pharmaceutical composition is configured for use in the treatment of cancer in a subject having a tumor.

14. (canceled)

15. A method for enhancing therapeutic efficacy of an immune checkpoint modulator in a subject, wherein the method comprises the administration of a Staphylococcal enterotoxin and the administration of an immune checkpoint inhibitor to the subject.
